(19) United States
(12) Patent Application Publication
(10) Pub. No.: US 2009/0123574 A1
(43) Pub. Date: May 14, 2009

(54) Compositions for the regression of spongiform pustules

(76) Inventors: N.B. Baktha Reddy, Chennai (IN); Vilambi Nrk Reddy, Trichy (IN); Anil Torgalkar, Gambury, NJ (US); N.R. Murugan, Trichy (IN); Senthil Kumar, Trichy (IN)

Correspondence Address: BREGEN TECHNICAL CONSULTANTS L.L.C., 154 OLD CLINTON ROAD, FLEMINGTON, NJ, 08822, US

(21) Appl. No.: 12/286,564

(22) Filed: Sep. 30, 2008

Related U.S. Application Data
(63) Continuation-in-part of application No. 11/395,944, filed on Mar. 31, 2006, now abandoned.

(51) Int. Cl. A61K 36/889; A61K 36/24; A61P 31/00

(52) U.S. Cl. 424/727; 424/725; 424/774; 424/779; 424/778; 424/777

(57) ABSTRACT

Herbal compositions that, when used topically in a therapeutically effective amount, are safe and effective for the regression of spongiform pustules in patients exhibiting chronic inflammatory skin disorders, such as psoriasis. The compositions, which can be formulated as ointments, oils, soaps and shampoos, comprise a non-aqueous herbal extract of Wrightia tinctoria, an herbal extract of Cocos nucifera, and pharmaceutically or cosmetically acceptable excipients suitable for topical use.

### CROSS-REFERENCE TO RELATED APPLICATIONS

The present application, filed under 35 U.S.C. 121, is a continuation in part of U.S. non-provisional patent application Ser. No. 11/395,944 entitled “Composition for Safe and Effective Regression of Spongiform Pustule,” filed on Mar. 31, 2006 and as such claims priority benefit under 35 U.S.C. 120 of this application. The disclosure of this application is herein incorporated by reference in its entirety.

### STATEMENT REGARDING FEDERALLY SPONSORED RESEARCH OR DEVELOPMENT

Not applicable.

### REFERENCE TO SEQUENCE LISTING, A TABLE, OR A COMPUTER PROGRAM LISTING COMPACT DISK APPENDIX

Not applicable.

### FIELD OF THE INVENTION

The present invention relates to herbal compositions for the regression of spongiform pustules. In particular, the present invention relates to herbal compositions for the regression of spongiform pustules in psoriatic lesions, psoriasis being one of the chronic inflammatory skin conditions presenting spongiform pustules.

### BRIEF DESCRIPTION OF THE BACKGROUND ART

The epidermis of the skin is a non-vascularized layer of the skin. Different factors can result in increased blood vessel formation in the papillary dermis and these blood vessels may sometimes extend into the epidermis resulting in clinically significant skin manifestations. For example, over-activated keratinocytes actively producing and secreting pro-angiogenic factors in the form of growth factors or cytokines can result in increased blood vessel formation in the papillary dermis, which may sometime extend into the epidermis. Epidermal microvascular proliferation ultimately leads to epidermal keratinocyte hyperproliferation, thickening of the epidermis with parakeratosis of the stratum corneum and inflammatory infiltrate around the blood vessels in the papillary dermis. The microvascular changes are also characterized by increased tortuosity of dermal capillary loops which precede the development of epidermal hyperplasia.

Histological studies, including immunocytochemistry, routine histology and electron microscopy have clearly established that alterations in the blood vessel formation of the skin discussed above are a prominent feature in chronic inflammatory skin conditions, including psoriasis, eczema, rheumatoid arthritis, burn granulations, and hypertrophic scars. In psoriasis, for example, there is a marked increase in the cutaneous blood active edge of the psoriatic plaque [Braverman I M, Yen A. Ultrastructure of the capillary loops in the dermal papillae of psoriasis. J Invest Dermatol 1977: 68: 53-60].

The regression of spongiform pustules in patients suffering from chronic inflammatory skin conditions provides the patient with benefits including but not limited to: reduced inflammation, reduced inflammatory edema, reduced erythema, reduced pain, reduced itching, help in resolving lesions and assistance in changing the epidermis to its normal non-vascularized state.

Spongiform pustules of Kagoj are multiocular pustules in the upper stratum malpighii within a sponge-like network made up of flattened keratinocytes. They are seen in psoriasis, Reiter's disease, geographic, tongue and, rarely, in candidiasis, among other chronic inflammatory diseases.

Numerous therapies in the field of allopathy medicine have been researched and developed to reduce spongiform pustules, especially in relation to psoriasis:
Treatment of psoriasis—Part 1—Topical Therapy and Phototherapy, Mark Lebwohl, MD, et al, American Academy of Dermatology October 2001 Vol 45 (4).
Treatment of psoriasis—Part 2—Systemic Therapies, Mark Lebwohl, MD, et al, American Academy of Dermatology, November 2001 Vol 45(5).
The immunological basis for the treatment of psoriasis with new biological agents. James. G. krueger, M.D, American Academy of Dermatology, June 2002 Vol 46(1) Pages 1-26.
   New psoriasis treatments based upon a deeper understanding of the pathogenesis of psoriasis vulgaris and psoriatic arthritis: Jeffrey. P. Callen et al, American Academy of Dermatology, August 2003 Vol 49(5) Pages 351-356

However, most of these therapies provide only temporary symptomatic relief and are either unsatisfactory or very expensive and are associated with either short term or long term undesired side effect profiles. [National Psoriasis Conference, Boston Plaza Hotel, Aug. 5-8, 2005, Boston, Mass., USA.]

It is well-known that herbal formulations often have fewer undesirable side effect profiles and hence provide a viable alternative therapy to manage the chronic inflammatory skin conditions that exhibit spongiform pustules. Research efforts to develop herbal formulations to treat these conditions have been on the rise and there is a continuing need to develop herbal formulations to treat spongiform pustules with minimal or no side effects:
Chopra, R. N., Nayar, S. C., and Chopra I. C., Glossary of Indian Medicinal Plants, C.S.I.R., P. 259, 1956.
Murugesa Mudaliar, K. S., Gunapadam (Material Medica) Vegetable Section, Govt. of TamilNadu, P. 527 (1969).
Venkatarajan, S., Sarabendra Vaithiya Muraigal, P. 160, 161 & 167 (1965).
Wealth of India, Raw Materials, Vol. X, P. 588-590, CSIR., New Delhi (1976).
Yugimuni Vaidya Chintamani (800) Stanza 494-518, B. Rathina Nayakar & Sons, Madras, India.
Nair, C. P. R., Kurup, P. B., Pillai, K. G. B., Geetha, A., and Ramiah, N., Effect of Nimbidin in Psoriasis, Indian Medical Journal, October 1978.

The invention provides herbal formulations, developed by a dermatologist, based on Wrightia tinctoria and an extract of Cocos nucifera which, when used as directed in therapeutically effective amounts, have been clinically proven to be safe and efficacious in reducing spongiform pustules in humans.

### SUMMARY OF THE INVENTION

It is an object of the present invention to provide a composition that, when used topically as directed, is suitable for the reduction of spongiform pustules. The composition can be formulated, for example, as an ointment, an oil, a soap or a shampoo. The composition is comprised of a non-aqueous herbal extract of Wrightia tinctoria, an herbal extract of Cocos nucifera and suitable pharmaceutically or cosmetically acceptable excipients designated for topical use in humans.

### BRIEF DESCRIPTION OF THE DRAWINGS

FIGS. 1 a and b: Micrographs of spongiform pustules before and after treatment with the herbal formulation respectively

### DETAILED DESCRIPTION OF THE INVENTION

The present invention relates to herbal formulations which unexpectedly provide statistically superior efficacy as compared to allopathy control formulations in the reduction of spongiform pustules (spongiform pustules of Kogoj) and are proven safe to use when used as directed. It is an object of the invention to provide an herbal composition that comprises a non-aqueous extract of Wrightia tinctoria, an herbal extract of Cocos nucifera, and pharmaceutically or cosmetically acceptable excipients suitable for topical use. The composition can be formulated as an ointment, an oil, a soap and a shampoo, and, when applied topically to the affected area in a therapeutically effective amount, it is effective in the treatment of spongiform pustules and is safe in humans. The spongiform pustules may be one of the symptoms presented in a case of psoriasis, especially generalized pustular psoriasis.

It is another object of the invention to provide the non-aqueous medium, which is a non-volatile oil, wherein the non-volatile oil is preferably a vegetable oil such as coconut oil, gingely oil (sesame oil), sunflower oil, corn oil, refined vegetable oil or a combination of oils. The non-volatile oil in the extract of the present invention comprises from about 80% to 99% by weight of the extract.

The herbal extract in the topical composition for the regression of spongiform pustules is derived from the Wrightia tinctoria plant, especially the leaves, twigs, flowers, fruits or a combination of these parts of the plant. Wrightia tinctoria is an apocynaceae tree growing throughout India. Its flowers are white and fragrant.

It is another object of the invention to provide a process for preparing the non-aqueous extract of Wrightia tinctoria. The non-aqueous extract is prepared at ambient temperature by cleaning and pulverizing the selected parts of the Wrightia tinctoria plant and soaking them in a non-aqueous oil medium containing coconut oil. Care should be taken to add sufficient oil medium to ensure that the plant material is completely submerged. The plant material/oil medium is then irradiated with a light source in the spectrum range of 300-1100 nm for a period of approximately 5 days. During this time the herbal ingredients are allowed to extract into the non-aqueous oil medium. At the end of the extraction, the oil medium is a purplish brown color. It is then filtered and the filtrate is stored for further processing as the non-aqueous herbal extract of Wrightia tinctoria.

Other herbal extracts may optionally be included in the formulation, including Melia azadirachta Linn oil (Neem oil), which has been documented to have beneficial skin effects [Nair et al., 1978]. The topical composition of the invention for the reduction spongiform pustules optionally comprises an extract of the active herbal ingredient mentioned above in the extraction medium in the amount from 1% to 20% by weight.

The herbal extract of Cocos nucifera in the composition of this invention is derived from the copra of the coconut. The copra of the coconut is dried and then processed by grinding and pressing to extract the oil, which is then purified and stabilized. The herbal composition of the present invention comprises the herbal extract (the oil) of Cocos nucifera present in the amount of 40% to 80% by weight.

It is an object of the invention to provide formulations for topical use by further compounding the compositions of the invention with ingredients mentioned herein to prepare formulations, including but not limited to, an ointment, an oil, a liquid soap and a shampoo.

The ointment formulation of the herbal composition of the invention suitable, when used topically in a therapeutically effective amount, for the reduction of spongiform pustules, includes pharmaceutically acceptable excipients such as beeswax, paraffin (liquid, soft and hard), and other standard ointment bases or their equivalents to optimize the use characteristics of the formulations, such as consistency and spreadability, as well as manufacturability and stability. The ointment composition comprises one or more of the excipients including beeswax, optimally present in the amount of 1 to 5% by weight; paraffin, optimally present in the amount of 5 to 40% by weight; and standard ointment bases, optimally present in the amount of 5 to 50%. by weight.

The oil formulation of the herbal composition of the invention suitable, when used topically in a therapeutically effective amount, for reduction of spongiform pustules, includes pharmaceutically acceptable excipients such as vegetable oil, animal oil, and synthetic oils such as mineral oil and liquid paraffin or their equivalents to optimize the use characteristics of the formulations, such as consistency and spreadability, as well as manufacturability and stability. Preferentially, the oil composition comprises excipients, such as coconut oil, present in the amount of 70 to 95% by weight.

The liquid soap formulation of the herbal composition of the invention suitable, when used topically in a therapeutically effective amount, for the reduction of spongiform pustules, includes pharmaceutically acceptable excipients, including but not limited to: water, surface active agents, thickeners and viscosity enhancers, foam boosters, and stabilizers to optimize the use characteristics, such as consistency, cleaning, spreadability and foaming, as well as manufacturability and stability. The liquid soap formulation of the present invention preferentially comprises excipients such as water present in the amount of 60 to 85% by weight; surface active agents present in the amount of 5 to 40% by weight; thickeners or viscosity enhancers present in the amount of 0.5 to 8% by weight; foam boosters present in the amount of 1 to 4% by weight; and stabilizers present in the amount of 0.5 to 2% by weight.

The shampoo formulation of the herbal composition of the invention suitable, when used topically in a therapeutically effective amount, for the reduction of spongiform pustules, includes pharmaceutically acceptable excipients, comprising water, surface active agents, thickeners or viscosity enhancers, foam boosters, and stabilizers to optimize the use characteristics such as consistency, cleaning, spreadability and foaming, as well as manufacturability and stability. The shampoo composition of the present invention preferentially comprises excipients including water present in the amount of 50 to 85% by weight; surface active agents present in the amount of 10 to 30% by weight; thickeners or viscosity enhancers present in the amount of 2 to 8% by weight; foam boosters present in the amount of 2 to 6% by weight; and stabilizers present in the amount of 0.5 to 2% by weight.

In addition, the ointment, oil, liquid soap and shampoo formulations of the herbal composition of the invention suitable, when used topically in a therapeutically effective amount, for the reduction of spongiform pustules, optionally comprise preservatives, coloring agents and fragrances as needed, wherein the preservatives, coloring agents and fragrances are present in the amount of 0 to 5 total weight %.

Safety and efficacy studies were conducted on subjects exhibiting spongiform pustules using topical formulations of the herbal composition of the present invention described above, containing a non-aqueous herbal extract of Wrightia tinctoria, an herbal extract of Cocos nucifera and pharmaceutically or cosmetically acceptable excipients. Patients suffering from chronic inflammatory skin conditions selected for the study exhibited spongiform pustules in the form of psoriatic lesions. Psoriasis is a representative example of spongiform pustules. The results are illustrated by the following example.

### EXAMPLE

### Safety and Efficacy Testing

Twenty patients were enrolled in a clinical study and were divided into two groups of 10 patients each. Group I was treated with the herbal formulation (see Table 1 for details) once daily and Group II was treated with an allopathy control formulation (see Table 2 for details) once daily. All patients recruited were screened and were determined to be suffering from problems with spongiform. All patients were psoriasis patients.

TABLE 1
Herbal Ointment Formula

| No | Ingredient | Quantity |
|---|---|---|
| 1 | Wrightia Tinctoria | 5% |
| 2 | Cocos Nucifera | 65% |
| 3 | Bees Wax | 6% |
| 4 | Liquid Paraffin | 24% |

TABLE 2
Dithranol Ointment
(Allopathy Control)

| No | Ingredient | Quantity |
|---|---|---|
| 1 | Dithranol | 1% |
| 2 | Standard Ointment Base | QS |

Assignment of patients to treatment groups was randomized as per standard statistical methods to minimize bias in the study. Patients were enrolled in the study on a first come, first served basis and assigned a subject number sequentially. The assignment of each patient to the treatment group was determined by the randomization list provided by the statistician.

### Efficacy Evaluation

Each patient voluntarily enrolled in the study and received the treatment for 8 weeks. Skin biopsies at the treatment site were taken from all patients at the beginning (T0) and end of the study (T8w) for histopathological evaluation. In addition, at the beginning (T0), end of treatment (T8w) haemogram analysis, liver function testing and renal function testing were done to document the safety profile of the treatments administered.

Histopathology of the skin biopsy was done by an expert pathologist and the spongiform pustule parameter was measured at visits T₀ and T_{8w}. The results of the spongiform pustule measurements were scored as follows:

(+)=3 representing heavy infiltrate in the epidermal cells

(±)=2 representing moderate infiltrate in the epidermal cells

(−)=1 representing mild infiltrate in the epidermal cells.

The spongiform pustule parameter represents the severity of infiltration in the epidermal cells. The more active the disease, the more severe the degree of infiltration.

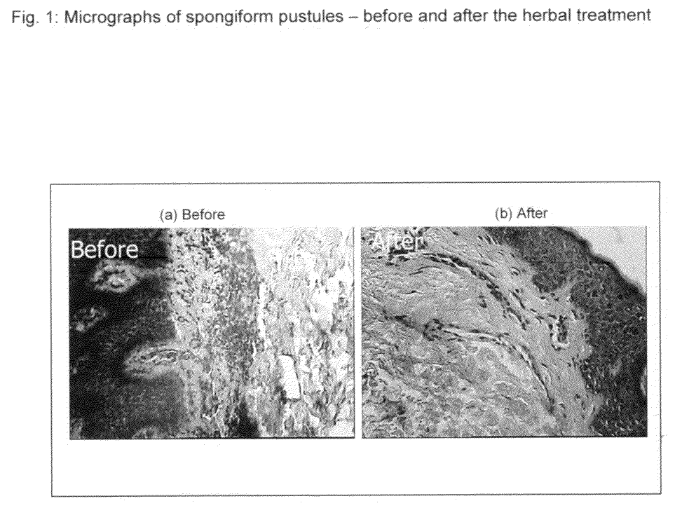

FIG. 1 presents photomicrographs of patients observed before and after the 8-week treatment with the herbal formulation of Wrightia tinctoria and Cocos nucifera. It is clear from the photographs that the treatment with the herbal formulation is very effective in regressing the spongiform pustules and in clearing the dermal infiltrate as compared to the condition of the patient prior to the start of treatment.

### Statistical Analysis of Results

Results of the statistical analysis of the spongiform pustule measurement data for the 2 treatment groups are presented in Table 3. A p-value of 0.05 is considered to be significant.

TABLE 3
Statistical Analysis of Histopathology Measurements for
Spongiform Pustule

| | Herbal | | Allopathy Control | | | |
|---|---|---|---|---|---|---|
| | (Group I) | | (Group II) | | | |
| | Mean | SD | Mean | SD | t | P-Value |
| Tow | 2.00 | 1.05 | 1.60 | 0.97 | 0.885 | 0.388 |
| T8w | 1.00 | 0.00 | 1.00 | 0.00 | — | — |
| Paired t | 3.00 | 1.05 | 1.964 | | | |
| Statistic | | | | | | |

| | | |
|---|---|---|
| Sig | 0.015 | 0.081 |
| (2 tailed) | | |

To examine the treatment effects, a t-test was performed with the data taken for the two groups at the beginning and the end of the treatment. No statistical significance was observed (p>0.05) for treatment effects on the spongiform pustule measurements at the beginning (p=0.388) and the end of the treatment (no difference in values between treatments).

To examine the time effects within each group, a paired t-test was done with data at the beginning and the end of treatment within each group. With the herbal group, there was a statistically significant time effect (p-values equal to 0.015) on the spongiform pustule measurements and it was found that the spongiform pustule values decreased with time, suggesting a positive response to the herbal treatment with time. However, with the allopathy control (Group II), no statistically significant time effect was found for the allopathy control formulation (p-value equal to 0.081).

The statistical data analysis clearly indicates that the herbal treatment for the regression of spongiform pustule is effective and is superior to the allopathy control formulation.

### Safety Evaluation

The safety of the use of the herbal formulation over the treatment period was evaluated by taking measurements of vital signs, haemogram measurements, liver function test (LFT) measurements, and renal function test (RFT) measurements and analyzing the data as a function of time.

The vital signs were measured 6 times during the treatment: T0, T1w, T2w, T4w, T6w, and T8w; the haemogram, the LFT and RFT measurements were made only at the beginning and end of the treatment (T0, T8w).

The results of the statistical analysis of the vital sign measurements (Systolic and Diastolic BP, pulse rate and respiratory measurements) are presented in Table 4. The BP was measured using a manual mercury sphygmomometer. The unit of measurement is mm of Hg. The pulse rate was measured (beats per minute) in the radial artery by palpating the artery with the middle, index and ring finger. The respiratory rate was measured by watching the expansion of the abdomen with each respiration and counting the expansions for one minute.

TABLE 4
Statistical Analysis of Vital Sign Measurements for Herbal Treatment.

| | | | | | | | Respiratory | |
|---|---|---|---|---|---|---|---|---|
| Time | BP-Systolic | | BP-Diastolic | | Pulse Rate | | Rate | |
| Points | Mean | SD | Mean | SD | Mean | SD | Mean | SD |
| 0w | 121.10 | 15.31 | 81.00 | 8.76 | 87.60 | 17.33 | 23.00 | 6.20 |
| T1w | 111.40 | 11.43 | 77.00 | 8.01 | 75.80 | 8.77 | 21.40 | 7.00 |
| T2w | 114.00 | 14.30 | 79.20 | 9.85 | 74.60 | 11.70 | 22.30 | 6.93 |
| T4w | 107.00 | 8.23 | 79.00 | 5.68 | 85.40 | 11.47 | 24.20 | 5.45 |
| T6w | 111.40 | 8.00 | 78.80 | 5.27 | 78.70 | 22.60 | 24.00 | 3.62 |
| T8w | 109.00 | 12.87 | 78.00 | 6.32 | 82.40 | 11.96 | 25.40 | 4.99 |
| Grand | 112.32 | 12.36 | 78.83 | 7.28 | 80.75 | 14.88 | 23.38 | 5.72 |
| Mean | | | | | | | | |

| | | | | |
|---|---|---|---|---|
| 1-Way | 1.674 | 0.317 | 1.273 | 0.612 |
| ANOVA | | | | |
| F-value | | | | |
| p-value | 0.157 | 0.901 | 0.289 | 0.691 |

A regular one-way ANOVA was also used to analyze the data at different time points for the vital signs measurements. The data clearly indicates that there were no statistically significant time effects on BP systolic measurements (p=0.157); BP diastolic measurements (p=0.901); pulse rate measurements (p=0.289) and respiratory rate measurements (0.691) with the herbal treatment. In summary, there is no statistically significant change in the vital sign measurements over time due to the treatment with the herbal formulation, suggesting no safety issues.

Results of the statistical analysis of the haemogram measurements [Total count of white blood cells (TC), differential white blood cells count as polymorphonuclear neutrophil (DC-P), lymphocytes (DC-L), eosinophils (DC-E) and haemoglobin (Hb)] are presented in Table 5. TC (Total count of white blood cells in the blood) was measured using a Neubauer Counting Chamber. The normal range for TC measurements is 4000-11,000 cells per cubicmillimetre. DC-P, which stands for the percentage of P-polymorphonuclear neutrophil, was measured using Neubauer Counting Chamber. The normal range for DC-P measurements is 55-65% of total white cell count. DC-L, which is the percentage of lymphocytes present, was measured using a Neubauer Counting Chamber. The normal range for DC-L Measurements is 30-40% of the total white cell count. DC-L was measured. DC-E, which is the percentage of eosinophils, was measured using the Neubauer Counting Chamber. The normal range for DC-E measurements is 1-7% of the total white blood cell count. DC-E was measured. HB, which is the haemoglobin measurements, was measured using the RA 50 Biochemical Analyzer and the normal range is 12-14 gms.

TABLE 5
Statistical Analysis of Haemogram Measurements for Herbal Treatment.

| Time | TC | | DC-P | | DC-L | | DC-E | | HB | |
|---|---|---|---|---|---|---|---|---|---|---|
| Points | Mean | SD | Mean | SD | Mean | SD | Mean | SD | Mean | SD |
| T0w | 7343 | 1589 | 57.30 | 2.95 | 37.90 | 1.79 | 4.80 | 2.90 | 13.02 | 1.72 |
| T8w | 8634 | 1105 | 58.90 | 2.69 | 37.10 | 2.38 | 4.00 | 2.49 | 12.95 | 0.94 |
| Paired | −1291 | 2279 | −1.60 | 3.78 | 0.80 | 3.22 | 0.80 | 3.77 | 0.075 | 2.13 |
| in differ | | | | | | | | | | |
| mean | | | | | | | | | | |

| | | | | | |
|---|---|---|---|---|---|
| Paired t | −1.791 | −1.340 | 0.784 | 0.672 | 0.100 |
| statistic | | | | | |
| Sig | 0.107 | 0.213 | 0.453 | 0.519 | 0.924 |
| (2- | | | | | |
| tailed) | | | | | |

To examine the time effects, a paired t-test was done with data taken at the beginning and end of treatment for each of the haemogram measurements. The data clearly indicates that there were no statistically significant time effects on TC measurements (p=0.107); DC-P measurements (p=0.213); DC-L measurements (p=0.453); DC-E measurements (p=0.519) and HB measurements (p=0.924) with the herbal treatment. In summary, there is no statistically significant change in haemogram measurements with time due to the treatment with the herbal formulation, suggesting no safety issues.

Results of the statistical analysis of the liver function test (LFT) measurements [serum glutamic oxalo acetic transaminase (SGOT), serum glutamic pyruvic transaminase (SGPT) and serum bilirubin] are presented in Table 6. SGOT, serum glutamic-oxalo acetic transaminase (international unit per liter), was measured at visits T₀ and T_{8w}. The normal range is 0-46 IU/L. SGPT, serum glutamic pyruvic transaminase (international units/liter) was measured at visits T₀ and T_{8W}. The normal SGPT ranges from 0 to 49 IU/L. The serum bilirubin was measured at visits T₀ and T_{8W}. The normal serum bilirubin ranges from 0.0 to 1.0 mg/dl.

TABLE 6
Statistical Analysis of Liver Function Test (LFT)
Measurements for Herbal treatment.

| Time | SGOT | | SGPT | | Serum Bilirubin | |
|---|---|---|---|---|---|---|
| Points | Mean | SD | Mean | SD | Mean | SD |
| T0w | 24.90 | 8.80 | 26.10 | 14.78 | 0.73 | 0.23 |
| T8w | 24.00 | 8.94 | 26.60 | 11.01 | 0.69 | 0.24 |
| Paired in | 0.90 | 10.97 | −0.50 | 11.24 | 0.035 | 0.31 |
| differ | | | | | | |
| mean | | | | | | |

| | | | |
|---|---|---|---|
| Paired t | 0.259 | 0.141 | 0.352 |
| statistic | | | |
| Sig | 0.801 | 0.891 | 0.733 |
| (2-tailed) | | | |

To examine the time effects a paired t-test was done with data taken at the beginning and the end of treatment for each of the LFT measurements. The data clearly indicates that there were no statistically significant time effects on SGOT measurements (p=0.801); SGPT measurements (p=0.891); and the serum bilirubin measurements (p=0.733) with the herbal treatment. In summary, there is no statistically significant change in LFT measurements with time due to treatment with the herbal formulation, suggesting no safety issues.

Results of the statistical analysis of the Renal Function Test (RFT) measurements [Serum Creatinine and Serum Urea] are presented in Table 7. Serum creatinine was measured at visits T₀ and T_{8W}. And the normal serum creatinine value ranges from 0.8 to 1.4 mg/dl. Serum urea was measured at visits T₀ and T_{8W}. And the normal serum urea value ranges from 10 to 50 mg/dl.

TABLE 7
Statistical Analysis of Renal Function
Test (RFT) Measurements for the Herbal
treatment.

| | | |
|---|---|---|
| | Serum | Serum |
| Time | Creatinine | Urea |

| Points | Mean | SD | Mean | SD |
|---|---|---|---|---|
| T0w | 1.06 | 0.22 | 32.40 | 17.50 |
| T8w | 1.08 | 0.18 | 25.49 | 7.75 |
| Paired in | −0.021 | 0.244 | 6.91 | 18.81 |
| differ | | | | |
| mean | | | | |

| | | |
|---|---|---|
| Paired t | −0.271 | 1.161 |
| statistic | | |
| Sig | 0.792 | 0.275 |
| (2-tailed) | | |

To examine the time effects, a paired t-test was done with data at the beginning and end of treatment for each of the RFT measurements. The data clearly indicates that there were no statistically significant time effects on serum creatinine measurements (p=0.792) and serum urea measurements (p=0.275) with the herbal treatment. In summary, there is no statistically significant change in RFT measurements with time due to the treatment with the herbal formulation, suggesting no safety issues.

It is clear from the histopathological examination and statistical analysis of the clinical data that the herbal formulations of the compositions of the present invention are very effective in the treatment of spongiform pustules and are superior to the allopathy control. In addition, the evaluation of haemogram, LFT and RFT test results clearly show that the herbal formula of the present invention is safe to use on humans.

Other modifications and variations of the present invention will become apparent to those skilled in the art from an examination of the above specification and examples. Therefore, other variations of the present invention may be made which fall within the scope of the appended claims even though such variations were not specifically discussed above.

I claim:

1. An herbal composition that, when used topically in therapeutically effective amounts, is effective for the regression of spongiform pustules, comprising:
an herbal extract of Wrightia tinctoria in a non-aqueous medium, an herbal extract of Cocos nucifera and pharmaceutically or cosmetically acceptable excipients for use in ointment, oil, soap and shampoo formulations.

2. The herbal composition according to claim 1, wherein the non-aqueous medium for the herbal extract is a non-volatile oil.

3. The herbal composition according to claim 2, wherein the non-volatile oil is a vegetable oil, selected from the group: coconut oil, gingely oil (sesame seed oil), sunflower oil, corn oil, and a refined vegetable oil.

4. The herbal composition according to claim 2, wherein the non-volatile oil is present in the extract in an amount of from 80% to 99% by weight of the extract.

5. The herbal composition according to claim 1, wherein the Wrightia tinctoria is obtained from at least one of the leaves, twigs, flowers, and fruit of the Wrightia tinctoria plant.

6. The herbal composition according to claim 5, wherein the herbal extract of Wrightia tinctoria is present in the non-aqueous medium in an amount of from 1% to 20% by weight.

7. The herbal composition according to claim 1, wherein the Cocos nucifera is extracted from the copra of the coconut.

8. The herbal composition according to claim 7, wherein the Cocos nucifera is present in the amount of 40% to 80% by weight.

9. An ointment formulation for the regression of spongiform pustules that, when used topically in therapeutically effective amounts, comprises the herbal composition according to claim 1 and pharmaceutically acceptable excipients.

10. An oil formulation for the regression of spongiform pustules that, when used topically in therapeutically effective amounts, comprises the herbal composition according to claim 1 and pharmaceutically acceptable excipients.

11. A liquid soap formulation for the regression of spongiform pustules that, when used topically in therapeutically effective amounts, comprises the herbal composition according to claim 1 and pharmaceutically acceptable excipients.

12. A shampoo formulation that, when used topically in therapeutically effective amounts, is suitable for the regression of spongiform pustules comprising the herbal composition according to claim 1 and pharmaceutically acceptable excipients.

13. A method for the regression of spongiform pustules which comprises administering to a subject in need thereof a formulation comprising a therapeutically effective amount of a composition according to claim 1, said formulation in a form chosen from the group: an oil, a shampoo, an ointment and a liquid soap.

14. An herbal composition for the regression of spongiform pustules in psoriatic lesions when used topically in therapeutically effective amounts, comprising:
an herbal extract of Wrightia tinctoria in a non-aqueous medium, an herbal extract of Cocos nucifera and pharmaceutically or cosmetically acceptable excipients for use in ointment, oil, soap and shampoo formulations.

15. The herbal composition according to claim 13, wherein the non-aqueous medium for the herbal extract is a non-volatile oil.

16. The herbal composition according to claim 14, wherein the non-volatile oil is a vegetable oil, selected from the group: coconut oil (Cocos nucifera), gingely oil (sesame seed oil), sunflower oil, corn oil, and a refined vegetable oil.

17. The herbal composition according to claim 14, wherein the non-volatile oil is present in the extract in an amount of from 80% to 99% by weight of the extract.

18. The herbal composition according to claim 14, wherein the Wrightia tinctoria is obtained from at least one of the leaves, twigs, flowers, and fruit of the Wrightia tinctoria plant.

19. The herbal composition according to claim 17, wherein the herbal extract of Wrightia tinctoria is present in the non-aqueous medium in an amount of from 1% to 20% by weight.

20. The herbal composition according to claim 18, wherein the Cocos nucifera is extracted from the copra of the coconut.

21. The herbal composition according to claim 19, wherein the Cocos nucifera is present in the amount of 40% to 80% by weight.

22. An ointment formulation for the regression of spongiform pustules in psoriatic lesions that, when used topically in therapeutically effective amounts, comprises the herbal composition according to claim 13 and pharmaceutically acceptable excipients.

23. An oil formulation for the regression of spongiform pustules in psoriatic lesions that, when used topically in therapeutically effective amounts, comprises the herbal composition according to claim 13 and pharmaceutically acceptable excipients.

24. A liquid soap formulation for the regression of spongiform pustules in psoriatic lesions that, when used topically in therapeutically effective amounts, comprises the herbal composition according to claim 13 and pharmaceutically acceptable excipients.

25. A shampoo formulation for the regression of spongiform pustules in psoriatic lesions that, when used topically in therapeutically effective amounts, comprises the herbal composition according to claim 13 and pharmaceutically acceptable excipients.

26. A method for the regression of spongiform pustules in psoriatic lesions which comprises administering to a subject in need thereof a formulation comprising a therapeutically effective amount of at least one composition according to claim 1, said formulation in a form chosen from the group: an oil, a shampoo, an ointment and a liquid soap.
